(19) United States
(12) Patent Application Publication
(10) Pub. No.: US 2006/0160231 A1
(43) Pub. Date: Jul. 20, 2006

(54) Linear analysis of polymers

(75) Inventors: Mark Nadel, Westborough, MA (US); John Harris, Foxboro, MA (US)

Correspondence Address: WOLF GREENFIELD & SACKS, PC;NULL, FEDERAL RESERVE PLAZA, 600 ATLANTIC AVENUE, BOSTON, MA, 02210-2206, US

(73) Assignee: U.S. Genomics, Inc., Woburn, MA (US)

(21) Appl. No.: 11/286,714

(22) Filed: Nov. 23, 2005

Related U.S. Application Data
(60) Provisional application No. 60/630,902, filed on Nov. 24, 2004.

(51) Int. Cl. G01N 33/00

(52) U.S. Cl. 436/85

(57) ABSTRACT

The invention relates to linear analysis of polymers and provides techniques to improve the amount and quality of information used to analyze polymers.

### RELATED APPLICATIONS

This application claims priority to U.S. Provisional Application Ser. No. 60/630,902 entitled “LINEAR ANALYSIS OF POLYMERS” filed Nov. 24, 2004, the entire contents of which are incorporated by reference herein.

### FIELD OF THE INVENTION

The invention relates to analysis of polymer sequence information, such as of biological polymers, and provides techniques and devices to improve the amount and quality of polymer information obtained.

### BACKGROUND OF THE INVENTION

Sequence analysis of polymers has many practical applications. Of great interest is the ability to sequence the genomes of various organisms, including the human genome. Specific sequences can be recognized with a host of sequence-specific probes such as oligonucleotides, peptides or proteins, and also synthetic compounds. In these sequence-specific approaches, there is sometimes a need to resolve the position of probes relative to one another, or to other features of the polymer, in order to generate a map of the polymer.

Linear analysis of polymers, such as DNA, may be accomplished by moving a detection zone over a fixed polymer, or by moving a polymer through a detection zone. These approaches make use of instrumentation and a detection signal to acquire information from the sequence-specific probes on the polymer when they are within the detection zone. For instance, fluorescence, atomic force microscopy (AFM), scanning tunneling microscopy (STM), as well as other electrical and electromagnetic methods, are suitable for capturing signals and thereby “reading” the sequence information of a polymer.

In certain circumstances, a probe on a polymer is not properly detected, thereby preventing proper analysis of the polymer. In some instances, an emission associated with a probe may not be strong enough with respect to the system noise level. In other systems where emission signals from probes are recorded as discrete data points representative of time intervals, it may be difficult to identify the specific location of a probe, particularly if its emission signal is spread over two or more discrete data points, or if the data points represent a significant passage of time. Still, in other situations, unbound probes may also be present in the detection zone, which can confuse the analysis of any probe-bound polymer within the detection zone at the same time. In other situations, a probe may not have properly hybridized with a polymer and thus might not be correctly positioned on the polymer.

### SUMMARY OF THE INVENTION

The invention is based, in part, on the discovery that multiple detection zones may be used during linear analysis of a polymer to acquire a greater amount of information when a polymer is passed there through. Some aspects of the invention increase the efficiency of polymer sequence analysis by increasing the amount of useful data that can be captured. Some aspects of the invention can be used to increase the signal-to-noise ratios (SNR) typical in some detection systems and, in doing so, can increase the quality of analysis that can be performed. Some aspects of the invention can also be used to increase the effective sampling rate of a polymer during linear analysis without drawbacks normally associated with an increased sampling rate, such as reduced signal-to-noise ratios. Aspects of the invention provide both methods and systems for analyzing polymers based on these discoveries.

According to an aspect of the invention, a method of analyzing at least one polymer is disclosed. The method comprises the acts of providing the at least one polymer with one or more labels thereon and providing a plurality of detection zones and instrumentation adapted to detect emission signals from labels that pass through the detection zones, each of the detections zones having a zone distance between an upstream edge and a downstream edge. Additionally, the method comprises the acts of passing the at least one polymer through at least a first and second detection zone of the plurality of detection zones at a velocity. Emissions are sampled from the first detection zone at a first sample interval as the at least one polymer passes through the first detection zone to create a first detection signal and emissions are sampled from the second detection zone at a second sample interval as the at least one polymer passes through the second detection zone to create a second detection signal. Also, the method comprises the act of combining the first and second detection signals together to create a combined signal used to analyze the at least one polymer. It is to be understood that labels disposed on polymers are detectable labels that are disposed on polymers usually via a sequence-specific probe that is itself bound to the polymer. Thus, the location of the label is usually indicative of the location of the probe which is in turn indicative of a particular sequence. Non-sequence specific detectable labels such as backbone labels, are discussed in greater detail herein.

According to another aspect, a method for increasing a number of sampling points of a single polymer passing through an interaction area having a first and a second detection zone is disclosed. The method comprises sampling emissions from the first detection zone as the polymer passes there through to provide a first set of discrete sample points and sampling emissions from the second detection zone as the polymer passes there through to provide a second set of discrete sample points. The method also comprises combining the first and second sets of discrete signal points to increase the number of sampling points of the polymer.

According to another aspect, a computer readable medium is disclosed that has computer readable signals stored thereon that define instructions that, as a result of being executed by a computer, instruct the computer to perform a method. The method is a method of increasing a number of sampling points of a polymer passing through a sampling area. The method comprises acts of sampling emissions from the first detection zone as the polymer passes there through to provide a first set of discrete sample points and sampling emissions from the second detection zone as the polymer passes there through to provide a second set of discrete sample points. The method also comprises acts of combining the first and second sets of discrete signal points to increase the number of sampling points of the polymer.

Some embodiments further comprise acts of sampling emissions from additional detection zones as the polymer passes there through to provide additional sets of discrete sample points and combining the additional discrete sample points with the first and second discrete sample points to increase the number of sampling points.

According to another embodiment, the method comprises acts of sampling emissions from additional detection zones of the plurality of detection zones as the at least one polymer passes through the additional detection zones to create additional detection signals. The method may also comprise an act of combining the additional detection signals with the first and second detection signals to create the combined signal used to analyze the at least one polymer.

In some embodiments, there are between 50 and 100 additional detection zones and additional detection signals.

In some embodiments, the at least one polymer is a single polymer. In other embodiments, the at least one polymer is a plurality of polymers.

According to some embodiments, any one of the at least one polymer is in a substantially similar position within each of the first and second detection zones when emissions are sampled.

According to some embodiments, any one of the at least one polymer is in a substantially similar position by being an equal distance from the upstream edge of the first detection zone and the upstream edge of the second detection zone when emissions are sampled.

Still, according to some embodiments, any one of the at least one polymer is in a substantially similar position due to either the first or second sample intervals being a factor of a transit interval between similar points within each of the first and second detection zones.

According to some embodiments, the first detection zone is adjacent to the second detection zone and the transit interval is substantially equal to the zone distance of the first detection zone. Still, in some embodiments, the transit interval is between 1 and 100 times the first sample interval.

According to yet some embodiments, any one of the at least one polymer is in a different position within each of the first and second detection zones when emissions are sampled.

In some embodiments, the first sample interval is different from the second sample interval such that any one of the at least one polymer is in a different position within each of the first and second detection zones when emissions are sampled.

In other embodiments, the velocity has velocity fluctuations that cause the first sample interval to be different from the second sample interval such that any one of the at least one polymer is in a different position within each of the first and second detection zones when emissions are sampled.

Still, in other embodiments, acquisition times associated with each of the first and second sample intervals are different such that any one of the at least one polymer is positioned differently within each of the first and second detection zones when emissions are sampled.

According to some embodiments, the first and second sample intervals are defined by the velocity multiplied by a first and second acquisition time, respectively. Additionally, the first and second acquisition times are out of phase with one another such that any one of the at least one polymer is in a different position within each of the first and second detection zones when emissions are sampled.

According to one method, a transit interval between similar points within each of the first and second detection zones is substantially equal to a multiple of either the first sample interval plus a constant or the second sample interval plus a constant such that any one of the at least one polymer is positioned differently within each of the first and second detection zones when emissions are sampled.

According to some other embodiments, the method further comprises acts of sampling emissions from a third detection zone at a third sample interval as any one of at least one polymer passes through the third detection zone to create a third detection signal, wherein any one of the at least one polymer is positioned substantially similarly within each of the first and third detection zones when emissions are sampled.

In one of the embodiments, the first detection zone is upstream of and adjacent to the second detection zone and the second detection zone is upstream of and adjacent to the third detection zone.

In another of the embodiments, the first detection zone is upstream of and adjacent to the second detection zone and the second detection zone is upstream of and separated from the third detection zone.

In some of these embodiments, the second and third detection zones are separated by two other detection zones from the plurality of detection zones.

According to some embodiments, each of the plurality of detection zones has a substantially similar zone distance.

According to some embodiments, combining the first and second detection signals comprises both aligning the first and second detection signals to one another, and summing the first and second detection signals together to create the combined signal.

According to some embodiments, the method comprises identifying an elapsed time between when one of the at least one polymer enters the first and the second detection zones and shifting the second detection signal by an amount of time substantially equal to the elapsed time to align the first and second detection signals.

According to some embodiments, the method comprises calculating a phase distance between where one of the at least one polymer enters the first and the second detection zones and shifting the second detection signal by the phase distance to align the first and second detection signals. In some embodiments, calculating the phase distance includes counting an elapsed time between when one polymer enters the first and second detection zones, and then multiplying the elapsed time by the velocity.

According to some embodiments, aligning the first and second detection signals comprises identifying a common element in each of the first and second detection signals and aligning the first and second detection signals by aligning the common element. In some of these embodiments, the common element is an emission from a backbone of the at least one polymer.

According to some embodiments, providing the plurality of detection zones comprises providing a linear CCD array having a plurality of pixels adapted to detect emission signals from the plurality of detection zones.

According to some embodiments, providing the plurality of detection zones comprises providing an initial timing detection zone and a final timing detection zone, wherein the initial timing and final timing detection zones are used to determine the velocity. In some of these embodiments, the initial and final detection zones are the first and second detection zones, respectively.

According to some embodiments, detection signals of the initial timing and final timing detection zones are detected by avalanche photo diodes. Still, in other embodiments, the detection signals of the initial timing and final timing detection zones are detected by photomultiplier tubes.

Additionally, according to one embodiment, passing the at least one polymer comprises passing the at least one polymer through a parallel row of the plurality of detection zones.

According to one embodiment, the method also comprises providing a microfluidic channel adapted to deliver a carrier fluid containing the at least one polymer through the plurality of detection zones.

In some embodiments, each of the plurality of detection zones comprises an area equal to one square micron.

In some embodiments, the at least one polymer is a peptide or a protein or a nucleic acid. Still in some embodiments, the nucleic acid is DNA or RNA. In some embodiments, the RNA is mRNA, siRNA or RNAi. The polymer may be other naturally occurring or non-naturally occurring polymers, such as polysaccharides.

In some of the embodiments, the velocity is between 0.1 and 20.0 mm/second.

According to one aspect, an apparatus is disclosed for the analysis of a polymer. The apparatus comprises a microfluidic channel having a first and a second end. The microfluidic channel is adapted to deliver a polymer disposed within a carrier fluid from the first to the second end. The apparatus also comprises an array of multiple detection zones disposed within the microfluidic channel and extending from the first end toward the second end, wherein the apparatus is adapted to detect emissions from the polymer as the polymer passes through the multiple detection zones to analyze the polymer.

In some embodiments, the array is a linear CCD array. In some of these embodiments, the CCD array comprises between 50 and 100 pixels. Still, in some embodiments, each of the pixels is associated with one of the multiple detection zones.

In some embodiments, the apparatus is adapted to create detection signals for each of the detection zones as the polymer passes there through. In some of these embodiments, the apparatus is adapted to combine the detection signals to analyze the polymer.

In some embodiments, the apparatus also comprises initial and final detection zones used to determine the velocity of the polymer as it is delivered through the microfluidic channel.

These and other aspects of the invention will be described in greater detail herein. Each of the aspects of the invention can be encompassed by various embodiments of the invention. It is therefore anticipated that each of the embodiments of the invention involving any one element or combinations of elements can be included in each aspect of the invention.

The phraseology and terminology used herein is for the purpose of description and should not be regarded as limiting. The use of “including”, “comprising”, or “having”, “containing”, “involving”, and variations thereof herein is meant to encompass the items listed thereafter and equivalents thereof as well as additional items.

### BRIEF DESCRIPTION OF THE FIGURES

The Figures are illustrative only and are not required for enablement of the invention disclosed herein.

Various embodiments of the invention will now be described by way of example, with references to the accompanying drawings.

FIG. 1 shows a schematic view of common components found in an embodiment of detection systems.

FIG. 2 shows a schematic view of a detection system having multiple detection zones disposed within an interaction station.

FIG. 3 shows a representation of a detection signal comprised of data points that represent emission intensity from a detection zone over different sample intervals, both plotted versus time and distance.

FIG. 4 shows multiple detection signals having data points created when a polymer is in a similar position in each of multiple detection zones. The signals each include emissions from the polymer and random noise. Also shown is the combination of the detection signals to reduce the impact of the noise.

FIG. 5 shows several representations of multiple detection zones, and identifies the transit interval between the detection zones and the sample intervals.

FIG. 6 shows multiple detection signals having data points created when a polymer was in a different position in each of multiple detection zones. Also shown is the combination of the detection signals to increase the effective sampling rate.

### DETAILED DESCRIPTION OF THE INVENTION

The methods and apparatuses of the present invention may be used to derive a greater amount of information from a polymer during linear analysis, particularly increasing the amount of information obtained per run, and/or per sample. In some embodiments, the additional information that is collected can increase the signal-to-noise ratio of systems used to analyze the polymer. In other embodiments, the additional information that is collected can more accurately define the position of features of the polymer by increasing an effective sampling rate of a signal used in analysis. These improvements may allow linear analysis to be performed with a greater degree of certainty, in a shorter time, and/or with a reduced number of polymers.

Some aspects of the present invention relate, in part, to a detection system having multiple detection zones that may each accept a labeled polymer, and a detector for detecting emissions from the detection zones as the polymer passes there through. In embodiments of the system, detection signals are created as the polymer passes through each of the detection zones. These detection signals may, in turn, be used to improve the analysis of the polymer through any of the various approaches discussed herein.

Some aspects of the present invention relate to improving the view of emissions associated with a polymer that is analyzed. To accomplish this in one illustrative embodiment, detection signals that comprise discrete data points, each representing emissions from a detection zone over a sample interval, are first created. In particular, the emissions are sampled over sample intervals when the polymer is in a substantially similar position within each of the detection zones. The detection signals are combined with one another such that discrete portions are aligned. In this manner, portions of the combined detection signal associated with the polymer are combined together and thus may be strengthened relative to other portions of the detection signal, such as those associated only with random system noise. In this manner the impact of random noise on the analysis is reduced, as it is likely that combining portions of the detection signal associated with random noise will not result in a strengthened signal, particularly as compared to those portions of the signal associated with the polymer.

Also, aspects of the present invention relate to identifying more accurately features of a polymer. In one illustrative embodiment, this is accomplished by creating detection signals such that their discrete data points represent a polymer when it is in a different position within each of two or more detection zones. These detection signals can be combined to produce a detection signal that has a higher effective sampling rate. That is, the combined signal may have data points that represent the polymer at more positions as it traverses the detection zones than the signals used to produce the individual detection signals. Creating a combined detection signal in this manner provides for more detailed analysis of a polymer without the drawback normally associated with increasing sampling rates of a detection system.

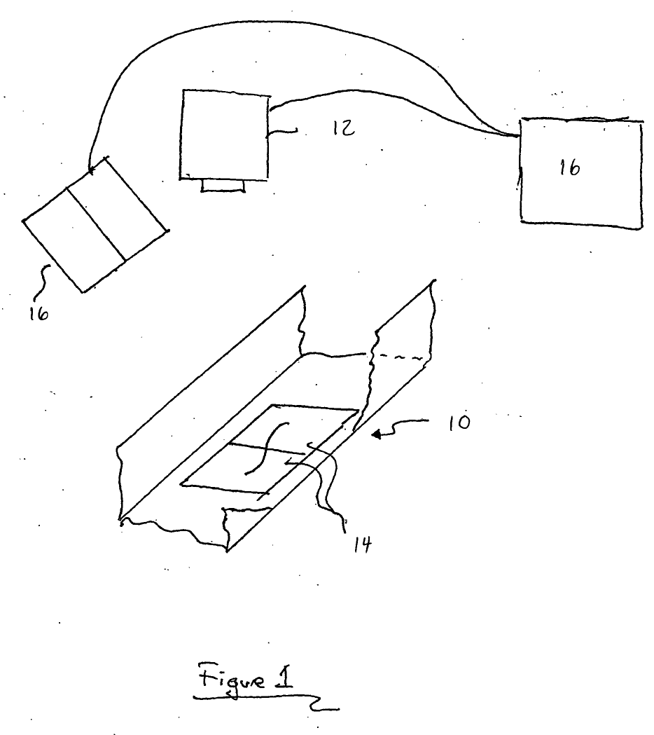

As shown in the Figures, and particularly FIG. 1, the basic components of many embodiments of detection systems include an interaction station 10 where a labeled sample is directed for detection or analysis. The labeled sample is a polymer or plurality of polymers bound to sequence-specific probes that are conjugated to detectable labels, or to non-sequence-specific labels such as backbone labels. An emitter 12, such as a laser, is projected into the interaction station 10 and may be used to excite features with which it interacts, such as the labels on the polymer or the polymer itself. One or more detection zones 14 are also present in the interaction station 10. Each detection zone is associated with a detector 16 that detects emissions from the zone, such as from a label or polymer within the detection zone. However, the detector typically also receives any other emissions from the detection zone, including noise. A detection signal that represents the emissions received is created by the detector and a downstream data processor 18. The data processor 18 may be used to analyze the detection signal along with other inputs, such as the spatial location of the polymer relative to the detector, the time when the emissions were detected, the spatial or temporal relationship between the various emissions that are detected, or other characteristics that may be used by various embodiments of detection systems as described herein.

Embodiments of the present invention are not limited to any particular type of detection system. However, many of the detection systems described herein have some components in common. In these systems, common terminology is used to describe components that may perform similar functions. As used herein, the term “interaction station” is used to define a portion of a detection system adapted to accept a sample for analysis. The sample may include a polymer but is not limited to the polymer alone. For example, the sample may include the polymer and a buffer solution along with any other elements contained within the buffer solution.

As used herein, the term “detection zone” is used to denote a volume within an interaction station from which emissions are received by a detector of the system. By way of example, in one embodiment of a detection system that uses confocal optical detection instrumentation, a detection zone is defined within the interaction station by a confocal aperture and an associated detector. In other optical detection systems, a detection zone is defined by a volume of the interaction station that provides optical emissions to a particular pixel or group of pixels of a CCD array. As is to be appreciated, the detection zones are not limited to these two particular embodiments, or to those associated with optical type detectors, and rather will embrace other forms.

As used herein, the term “upstream edge” refers to the side of a detection zone that, in typical detection system operation, first receives a polymer to be analyzed as the polymer and the detection zone are moved relative to one another. As used herein, the term “downstream edge” refers to the side of a detection zone where the polymer exits the detection zone as the polymer and detection zone are moved relative to one another. Also, as used herein, “zone distance” refers to the distance between the upstream and downstream edge of a detection zone. As is to be appreciated, the upstream edge and the downstream edge may be formed of any boundary that a detection system has, such as a straight line or an arc that defines an edge of a particular detection zone. As is also to be appreciated, in some embodiments like those having edges defined by an arc, the zone distance may not be constant for all paths across the detection zone. In such cases, the zone distance may be calculated as the average distance across the detection zone.

As used herein, the term “detector” is used to denote a component of a detection system that receives emissions from a detection zone. The information received by the detector may, in turn, be used by a data processor to understand whether a polymer is present in a detection zone and/or to identify the characteristics of a polymer present in the detection zone. Some examples of detectors that may be used in optical detection systems include Charge Coupled Devices (CCD's), avalanche photo diodes, and photomultiplier tubes. These particular embodiments of detectors may be adapted to receive photon emissions from a detection zone, and to convert the emissions into an electrical signal having discrete data points representing the number of photons received during a given sample interval. This signal may then be passed to a downstream data processor for further manipulation or analysis. As is to be appreciated, embodiments of the invention may use other types of detectors as the invention is not limited to the examples given above, or the manner in which these exemplary detectors operate.

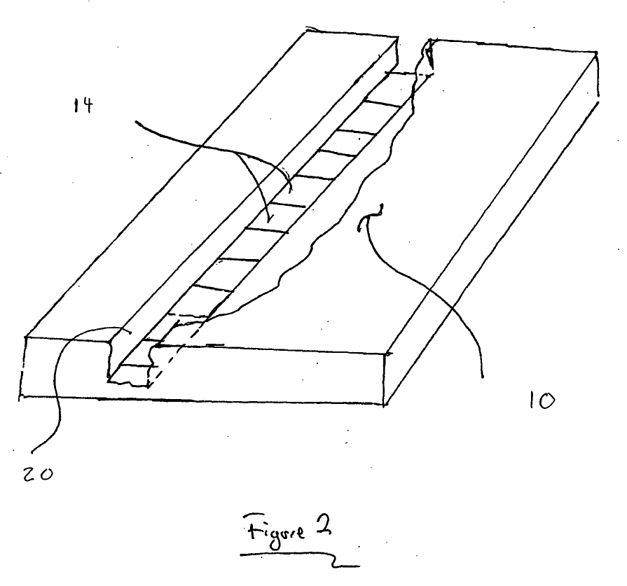

As represented by FIG. 2, the detection system in one illustrative embodiment of the invention has a plurality of detection zones 14 located within an interaction station 10. The interaction station in this embodiment is disposed within a microfluidic channel 20 that directs a carrier fluid containing a labeled polymer through each of the detection zones in a serial manner. A laser light illuminates the contents of the detection zones, such as labels on the polymer or the polymer itself. Emissions from the illuminated contents of the detection zones are then collected by pixels of a linear CCD array, each associated with one of the detection zones 14 as shown in FIG. 2, or other types of detectors. The emissions unto each pixel are used to create separate detection signals for each corresponding zone.

Although embodiments of the present invention may have two detection zones used to create two separate detection signals for any polymer, as shown in FIG. 1, the invention is not limited to any particular number of detection zones or detectors. By way of example, FIG. 2 shows a portion of a detection system having 100 detection zones. Furthermore, embodiments are not limited to any particular arrangement of detection zones within an interaction station. A detection zone or zones may entirely cover an interaction station, a sub portion of the interaction station, or may even extend beyond the interaction station. Individual detection zones may be overlapped, either partially or entirely with other detection zones. Detection zones may even share common upstream and downstream edges in some embodiments or may be separated from other detection zones completely, as the present invention is not limited to any particular configuration of the detection zones within an interaction station.

Polymers may be analyzed using a single molecule analysis system (e.g., a single polymer analysis system). A single molecule detection system is capable of analyzing single molecules separately from other molecules. Such a system may be capable of analyzing single molecules either in a linear manner and/or in their totality. As a polymer is analyzed, the detectable labels attached to it are detected in either a sequential or simultaneous manner. In some embodiments, the polymer may be capable of inherently generating signals and these would also be captured by the systems and methods described herein. When detected simultaneously, the signals usually form an image of the polymer, which may or may not yield information regarding distances between labels. For example, if the method employs FRET analysis, presence or absence of a signal indicates distance between FRET donors and FRET acceptors. However, if the analysis is a not FRET based, then presence or absence of a signal may in some embodiments reveal simply whether a particular label (and thus potentially a sequence) is present or absent.

A linear polymer analysis system is a system that analyzes polymers in a linear manner (i.e., starting at one location on the polymer and then proceeding linearly in either direction therefrom). In certain embodiments in which detection is based predominately on the presence or absence of a signal, linear analysis may not be required. However, there are other embodiments embraced by the invention which would benefit from the ability to analyze polymers linearly. These include applications in which the sequence of the polymer or relative position of different landmarks on a polymer is desired to be known. When detected sequentially, the signals may be viewed in histogram (signal intensity vs. time). The histogram data can then be translated into a map with knowledge of the polymer velocity. It is to be understood that in some embodiments, the polymer is attached to a solid support, while in others it is free flowing. In either case, the velocity of the polymer as it moves past, for example, an interaction station or a detector, will aid in determining the position of the labels, relative to each other and relative to other detectable landmarks that may be present on the polymer.

Accordingly, the analysis systems useful in the invention may deduce the total amount of label on a polymer, and in some instances, the location of such labels. The ability to locate and position the labels allows these patterns to be superimposed on other genetic maps in order to orient and/or identify the regions of the genome being analyzed.

An example of a suitable system is the GeneEngine™ (U.S. Genomics, Inc., Woburn, Mass.). The Gene Engine™ system is described in PCT patent applications WO98/35012 and WO00/09757, published on Aug. 13, 1998, and Feb. 24, 2000, respectively, and in issued U.S. Pat. No. 6,355,420 B1, issued Mar. 12, 2002. The contents of these applications and patent, as well as those of other applications and patents, and references cited herein are incorporated by reference in their entirety. This system is both a single molecule analysis system and a linear polymer analysis system. It allows, for example, single nucleic acids to be passed through an interaction station in a linear manner, whereby the nucleotides in the nucleic acid are interrogated individually in order to determine whether there is a detectable label conjugated (directly or indirectly) to the nucleic acid. Interrogation involves exposing the nucleic acid to an energy source such as optical radiation of a set wavelength. The mechanism for signal emission and detection will depend on the type of label sought to be detected, as described herein.

Other nucleic acid analytical methods which involve elongation of DNA molecules can also be used in the methods of the invention. These include fiber-fluorescence in situ hybridization (fiber-FISH) (Bensimon, A. et al., Science 265(5181):2096-2098 (1997)). In fiber-FISH, nucleic acid molecules are elongated and fixed on a surface by molecular combing. Hybridization with fluorescently labeled probe sequences allows determination of sequence landmarks on the nucleic acid molecules. The method requires fixation of elongated molecules so that molecular lengths and/or distances between markers can be measured. Pulse field gel electrophoresis can also be used to analyze the labeled nucleic acid molecules. Pulse field gel electrophoresis is described by Schwartz, D. C. et al., Cell 37(1):67-75 (1984). Other nucleic acid analysis systems are described by Otobe, K. et al., Nucleic Acids Res. 29(22):E109 (2001), Bensimon, A. et al. in U.S. Pat. No. 6,248,537, issued Jun. 19, 2001, Herrick, J. et al., Chromosome Res. 7(6):409:423 (1999), Schwartz in U.S. Pat. No. 6,150,089 issued Nov. 21, 2000 and U.S. Pat. No. 6,294,136, issued Sep. 25, 2001. Other linear polymer analysis systems can also be used, and the invention is not intended to be limited to solely those listed herein.

As used herein, the term “detection signal” is used to denote a signal that is created to represent all or a portion of the emissions from a detection zone within a detection system. An example from an optical detection system is a detection signal that may be created from photons emitted by the contents of a detection zone, including those of a labeled polymer or the polymer itself. The emissions may be recorded in terms of emission intensity versus time in some embodiments. Specifically, the signal may comprise data points representative of a count of photons received from a detection zone over a period of time. In other embodiments, the detection signal may comprise a representation of signal intensity versus position for all emissions that a detector receives as a detection zone is moved about an interaction station. As is to be appreciated, detection signals are not limited to either of these two described examples, as those of skill may appreciate that other forms of detection signals may be used in detection systems.

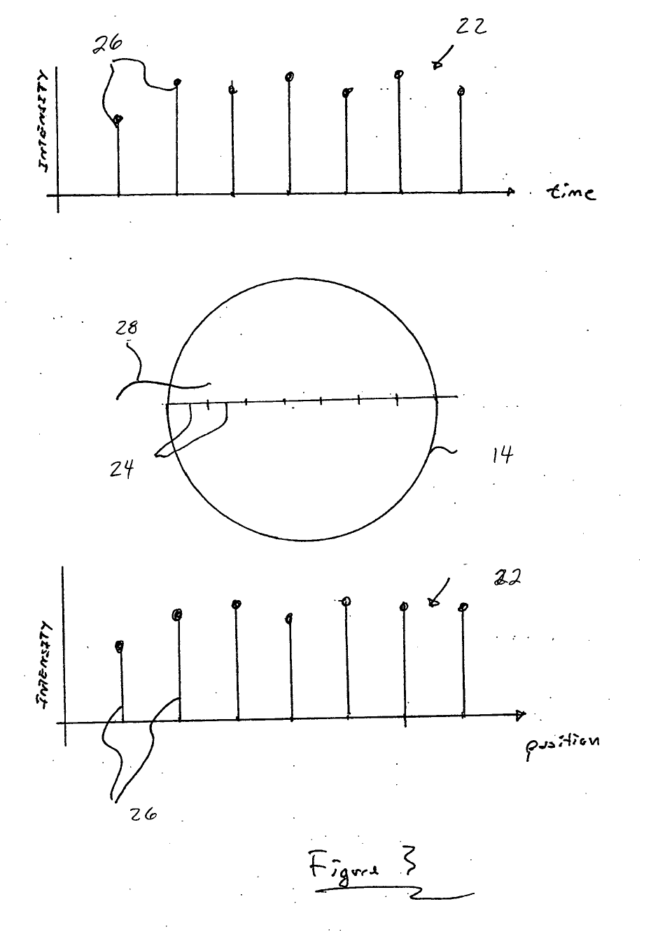

As discussed briefly above, detection signals, at least in optical detection systems, may comprise counts of photons emitted by labels and other contents of a detection zone. Generally, the photon counts are collected over time (or position) to produce a detection signal 22 of intensity versus time (or position), like that shown in FIG. 3. As shown here, the photons are collected and counted during a sample interval 24, after which the count resets and a new count is begun. This process is referred to herein as “sampling”. In such an embodiment, sample interval 24 denotes the amount of time that passes between the beginning and end of a count. The photon counts associated with the sample intervals are represented by the individual data points 26 of the detection signal in FIG. 3. It is to be appreciated that while FIG. 3 shows a graph of signal intensity versus time, a detection signal may be expressed in different terms. By way of example, a detection signal may be expressed as signal intensity versus position, representing the position of a polymer 28 with respect to the detection zone as they move relative to one another. This is also represented in a separate graph shown in FIG. 3. In such systems, the term “sample interval” may denote a distance, rather than a period of time, as the term is generic in this sense. In other embodiments, a detection signal expressed in terms of intensity versus time may be converted to the spatial domain with knowledge of the relative velocity between the detection zone and a polymer.

Detection signals created from different detection zones can be combined to improve the amount of information obtained from a polymer. According to one illustrative embodiment, the signals produced by the detectors are combined such that data points are aligned with one another. Here, the data points may represent a polymer or polymers at a substantially similar position within different detection zones. As previously discussed, combining the detection signals may serve to strengthen the portions of the detection signal that are associated with emissions of the labeled polymer relative to other portions of the detection signals, such as those associated only with noise in the system. It is to be appreciated that not all embodiments combine detection signals in this manner or even to accomplish such an effect, as the present invention is not limited in this regard.

Detection systems frequently include system noise, which can complicate analysis performed by the system. As used herein, “noise” denotes contributions to a detection signal that are not related to a labeled polymer. Various factors may contribute to the noise level of the system, such as imperfections in a detector, imperfections in instrumentation associated with a detector or a data processor, impurities in the sample, unbound labels and probes, foreign particles in the sample, or even a carrier fluid of the sample. In some instances, the presence of noise within a detection signal can mask portions of the detection signal that are associated with a polymer. Still, in other instances, noise may falsely indicate the presence of a polymer, or a particular feature of a polymer.

The noise in many embodiments can be characterized by an average noise level, which is the average intensity of noise detected in a system when a control sample is present in a detection zone (i.e., a sample containing no labeled polymers or unlabeled polymers, as the case may be). As used herein, the term “signal-to-noise ratio” (SNR) refers to the ratio between the intensity of emissions associated with a labeled (or unlabeled) polymer and the average intensity of the system noise level. As may be appreciated, it is generally desirable to have a higher signal-to-noise ratio, as it may facilitate identifying emissions associated with a labeled polymer or particular features of a polymer.

To help minimize the impact of system noise on the detection system, some embodiments require a threshold level of emissions, such as a particular number of photons, to be collected within a given sample interval before any photons are acknowledged and recorded. Otherwise, the data point representing that sample interval may be set to zero. The threshold level in some illustrative embodiments can be set at or above the noise level of the system to prevent noise from inadvertently being interpreted as the presence of a polymer whether labeled or not.

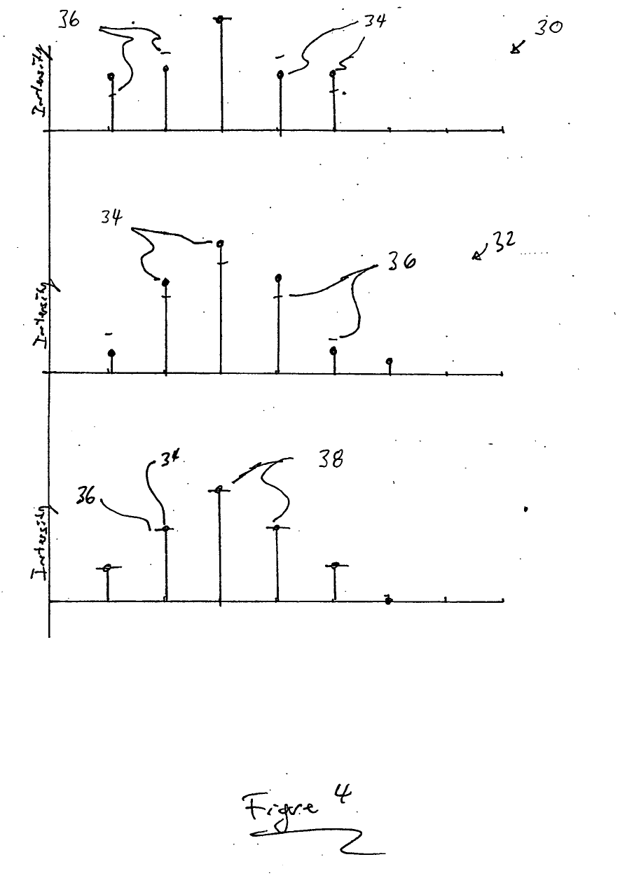

As mentioned above, according to some illustrative embodiments of the invention detection signals are combined to reduce the impact of noise on emissions from the labeled polymer, effectively increasing the signal-to-noise ratio. FIG. 4 provides an illustration of how this is accomplished, according to one embodiment. A first detection signal 30 and a second detection signal 32 are created, each associated with a first and a second detection zone and the passage of a labeled polymer there through. Each of the first and second detection signals has data points that represent the polymer being positioned substantially similarly within each of the first and second detection zones. In addition to having components that represent emissions from the labeled polymer, the detection signals have contributions from random noise in the system. This is shown in FIG. 4 by the theoretical data points 34 that represent measurements taken in a noiseless system, and data points 36 that represent measurements that include noise components. When the signals are created and combined with their data points aligned, the strength of emissions associated with labeled polymer is increased, particularly relative to random events in the detection signals. This is represented by the combined signal 38 in FIG. 4. As may be appreciated, when portions of multiple detection signals associated with random noise are combined, the probability of the noise components canceling each other out, or at least being attenuated by the combination, is greater than the probability that the effect of the noise components will be increased. However, the presence of non-random events in the detection signal, such as the emissions associated with the labeled polymer, are not attenuated but rather reinforced or strengthened by the combination.

According to some embodiments, combining more detection signals in the above described method will further improve the signal-to-noise ratio, thus further reducing the effects of noise in the detection system. In one embodiment as represented in FIG. 2, approximately one hundred detection signals are created from one hundred separate detection zones disposed within an interaction station and may be combined to improve the signal-to-noise ratio. In particular, this illustrative embodiment uses a linear CCD array having 100 pixels disposed in a row, where each pixel is associated with a detection zone in the interaction station. However, it is to be appreciated that the present invention is not limited in this regard, as any number of detection zones and detection signals may be used.

Certain aspects of the present invention, such as those used to improve the signal-to-noise ratio, may be used in the analysis of a single polymer or multiple polymers. As is to be appreciated, analysis schemes for some detection systems may include analysis of multiple copies of the same polymer (i.e., an amplified nucleic acid population). These multiple identical polymers may be simultaneously analyzed. As is also to be appreciated, some analysis schemes may favor or require that analysis be performed on a single polymer for which no additional identical copies are available in real time. By way of example, some analysis schemes may need to be performed in an amount of time that does not permit prior polymer amplification to be performed.

As mentioned above, some detection systems may analyze several identical copies of a polymer to create multiple detection signals for each similarly labeled polymer. As with systems that analyze only a single copy of a polymer, detection signals associated with each of the distinct, yet identical polymers may be combined to improve the signal-to-noise ratio of the combined detection signal. As is also to be appreciated, as the number of detection zones and corresponding detection signals is increased, the number of polymer copies needed to create the same number of detection signals is reduced. In this manner, having multiple detection zones and corresponding detection signals can allow analysis to be performed on a single labeled polymer, instead of requiring multiple polymers to achieve a particular signal-to-noise ratio.

Some aspects of the present invention can improve the signal-to-noise ratio of a detection system without trade-offs associated with other techniques for improving the signal-to-noise ratio. Techniques that may alter the signal-to-noise ratio, yet may also impact other aspects of the detection system include altering the zone distance, altering the sample interval, altering the acquisition time, and/or changing the relative velocity between the polymer and the detection zone, are described in greater detail below.

As previously described, the sample interval is a period of time or distance over which emissions from a detection zone are collected. As is to be appreciated, when the sample interval is decreased in an optical detection system, with all else constant, fewer photons will be collected from any labeled polymer passing through a detection zone during a given sample interval. In some instances, this may cause a detection signal from a labeled polymer to fall below the threshold noise level of the system, which can prevent the polymer from being detected or from being detected properly. Increasing the sample interval typically has the opposite effect. Increasing the sample interval to raise the signal level above the threshold level in such a scenario may also cause some effects that are not desirable. By way of example, as the sample interval increases, the position of any labels on a polymer represented by the detection signal may become more difficult to discern, as the sample interval now represents an increased area and/or time. The trade-offs associated with increasing and decreasing the sample interval and other characteristics of detection systems are discussed in U.S. patent application Ser. Nos. 10/246,779 and 10/762,207, each hereby incorporated by reference in its entirety.

As discussed previously, sample interval may be the distance or time that a polymer travels as represented by a single data point of a detection signal. In cases where sample interval represents a distance, the sample interval may be converted to a time by dividing the sample interval by the relative velocity between the polymer and the detection zone. “Acquisition time,” as used herein, denotes the amount of time associated with a sample interval of a detection signal, regardless of whether the sample interval is expressed as a time or a distance. That is, in the case of an optical detection system, it represents the amount of time that passes before a photon count from a detection zone is reset.

Another parameter of detection systems that may affect the amount of information collected within a given sample interval is relative velocity. Embodiments of the present invention allow the amount and quality of information collected to be improved, without requiring a factor like the relative velocity to be altered, although in some embodiments it may be desirable to alter the relative velocity. A polymer that moves through a detection zone faster will reside within the detection zone for a shorter period of time and any detectable portions of the polymer or labels thereon will emit fewer photons (in an optical embodiment) as they pass through the detection zone. As previously mentioned, it is generally preferred to receive more photons from any given labeled polymer to help distinguish time detection signals from the noise level of the system. As such, a slower relative velocity may be generally preferred for detection signal quality reasons. However, a slower relative velocity generally means that the overall analysis may take longer to complete. Since it may be preferable to complete an analysis in a shorter timeframe, there is typically a trade-off between relative sample velocity, which directly impacts the speed at which the detection system may operate, and the quality of the data collected (i.e., the strength of the signal collected relative to the noise level in the system). Aspects of the present invention may allow the analysis time to be reduced without sacrificing the data quality.

As discussed herein, according to some embodiments of the invention, the detection system is adapted such that a labeled polymer is positioned substantially similarly within different detection zones during sample intervals associated with each of the detection zones. That is, a polymer or multiple polymers are each in a substantially similar position within each detection zone when the sample interval begins, and when the sample interval ends. By way of illustrative example, in one embodiment, a labeled polymer just entering the first detection zone at the beginning of a sample interval may also just enter the second detection zone at the beginning of another sample interval. In this regard, sampling of the detection zone, or emissions from the detection zone occurs when the polymer is positioned substantially similarly.

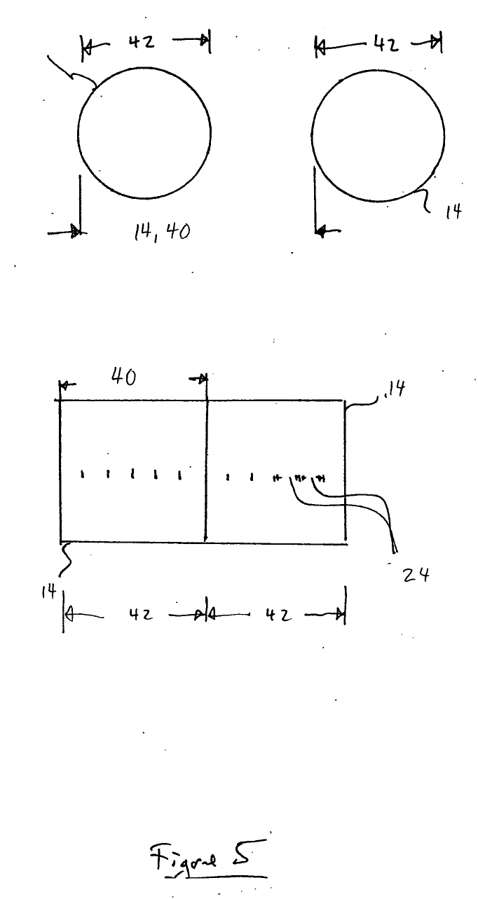

According to some illustrative embodiments, the detection system may be designed such that a “transit interval” between detection zones is a multiple of a sample interval in the system to allow a labeled polymer to be in a substantially similar position when emissions from detection zones are sampled. As used herein, the term “transit interval” denotes the distance between substantially similar points within different detection zones. In particular, transit interval usually refers to the distance between substantially similar points in adjacent detection zones, although it is not limited in this manner. Transit interval may be measured in terms of distances or in units of time when divided by the relative velocity between a polymer and the corresponding detection zones. FIG. 5 schematically represents transit interval 40 and sample interval 24 in several different embodiments of detection systems, each adapted to have a polymer positioned substantially similarly within detection zones 14 as emissions are sampled. In a first embodiment, transit interval 40 is depicted between two detection zones 14 that have different zone distances 42 and that are separated from one another. In this particular embodiment, the transit interval 40 is equal to the sample interval 14. In a second embodiment, as is also represented in FIG. 5, the detection zones 14 are adjacent to one another and have the same zone distance 42. Here, the sample interval 14 is a factor of the transit interval 46, such that a polymer may be in several substantially similar positions in the detection zones 14 when emissions are sampled.

To be in a substantially similar position within a detection zone, as this phrase is used herein, denotes that the labeled polymer is positioned in one detection zone within plus or minus ten percent, and more preferably plus or minus five percent of its position in another detection zone, using the same measure, when one sample interval ends and the next sample interval begins. Also, as used herein, intervals that are said to be “substantially equal” denotes that the intervals are within plus or minus ten percent, and more preferably plus or minus five percent, and even more preferably plus or minus one percent of the same size as one another, using the same measure. The measure for these characteristics in different embodiments of systems may not always be the same. By way of example, in some embodiments the polymer may be positioned a substantially similar distance from an upstream edge of two different detection zones as emissions are sampled. In other embodiments, the polymer may have traversed an equal proportion of the zone distance of two different detection zones when emissions are sampled. In such embodiments, the zone distance of the different detection zones may not be substantially similar.

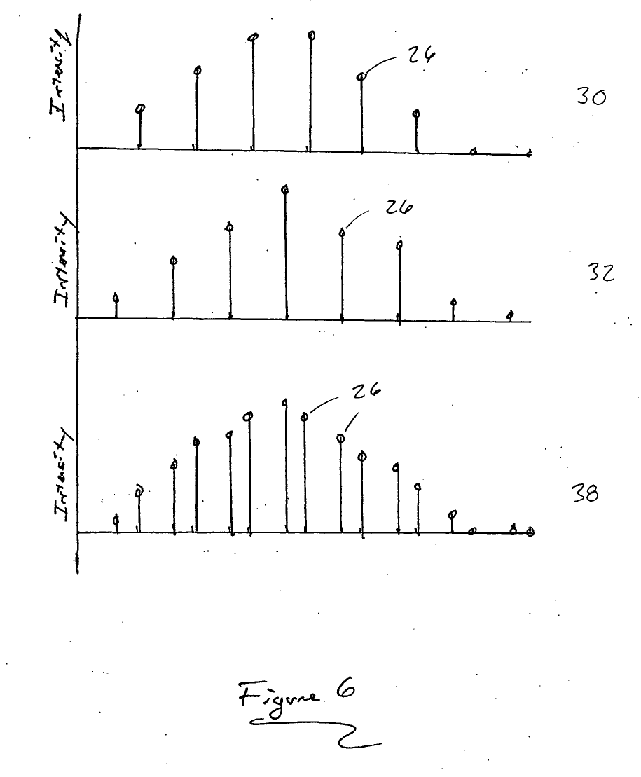

According to another illustrative embodiment of the invention, a labeled polymer may be in a different position within each of multiple detection zones when emissions are sampled. In such embodiments, detection signals associated with each of the detection zones may be combined to produce a detection signal with a higher effective sampling rate, as illustrated in FIG. 6. As may be appreciated, detection signals 22 comprise data points 26 that represent emissions from a detection zone 14 during a given sample interval 24. The collection of data points in a detection signal can provide a view of the emissions of a labeled polymer at discrete points, as it passes through the detection zone. In some embodiments, it is desirable to increase the resolution of this view of the polymer. That is, it may be desirable to increase the number of data points such that features of the polymer or one of its labels may be viewed in greater detail. In one embodiment, viewing these emissions in greater detail may allow the position of a label on a polymer to be identified with greater precision. For example, having more data points, or an increased effective sampling rate, may allow the peak or the start or end of the emissions associated with a polymer to be more readily identified, which may allow detection systems to define more accurate positions of a label on a polymer.

As is to be appreciated, various approaches may be employed to sample emissions from detection zones when a polymer is in a different position in the detection zones. By way of example, the acquisition times associated with the detection signals may be of different lengths of time and/or may be phased with respect to one another. As used herein, the term “out of phase” when used to describe acquisition times means that the acquisition times do not start and end at the same time. The relative velocity between the detection zones and the polymer may change between different detection zones. Still, in some embodiments, transit intervals between various adjacent detection zones of the system may be different thereby causing a polymer to be in a different position in the various detection zones as emissions are sampled. Still other approaches may be taken, as aspects of the invention are not limited to the above listed methods.

As previously described, acquisition time refers to the amount of time associated with a sample interval of a detection signal. To cause a polymer to be positioned differently within detection zones of a system, one or more of the detection zones may have acquisition times that are phased with respect to one another. That is, the acquisition times may be of the same duration, but begin and end at different times with respect to one another. This is represented in FIG. 6 by the first detection signal 30, and the second detection signal 32, that correspond to the first and second groups, respectively. In this manner, the sample intervals, and thus the data points of the associated detection signals, may represent a polymer in a different position of the associated detection zones where the sample intervals of the different detection curves will begin and end at different times. According to one embodiment, a portion of a plurality of detection zones has acquisition times that are in phase with one another while another portion of the detection zones has acquisition times that are phased with respect to those of the first portion of detection zones. In this manner, detection signals are created that both represent polymers in substantially similar positions of respective detection zones and in different positions of respective detection zones as emissions are sampled. Combining such detection signals can cause the resulting combined detection signal 38, as shown in FIG. 6, that has both an increased signal-to-noise ratio and an increased effective sampling rate. However, it is to be appreciated that other approaches can also allow detection systems to embody each of these benefits, as the invention is not limited in this regard.

As is to be appreciated, the acquisition times of different detection zones may also be different from one another to allow emissions to be sampled from the detection zones when a polymer is positioned differently therein. In some of these embodiments, the different acquisition times may be multiples of one another such that, periodically, emissions are sampled while a polymer is in a substantially similar position within each of the detection zones. However, in other embodiments the different acquisition times may not be multiples of one another, as the invention is not limited in this manner.

According to other embodiments of the invention, a labeled polymer may be positioned differently in different detection zones when emissions are sampled by varying the velocity with which the polymer passes through each of the detection zones. In this manner, the polymer may be in a different position as emissions are sampled from the detection zones. In some embodiments, particularly those where multiple polymers are passed in a carrier fluid through a microfluidic channel, the natural fluctuations in the flow velocity of the carrier fluid may cause this to occur between the different polymers. In other embodiments using microfluidic channels, velocity gradients established by features, like those described in application Ser. No. 10/821,664, titled “Advanced Microfluidics”, filed on Apr. 9, 2005, now published as U.S. Patent Application 2005-0112606, hereby incorporated by reference in its entirety, may cause the velocity to vary from one detection zone to another. Still, in other embodiments, the detection zone may be moved at a changing velocity relative to a stationary sample, as the invention is not limited to one particular method for changing relative velocity between a polymer and the detection zone.

As mentioned briefly above, different aspects of the invention may be combined in some illustrative embodiments. For instance, in one embodiment, emissions may be sampled from a first portion of a plurality of detection zones when a polymer is in a substantially similar position within the detection zones. Emissions may also be sampled from a second portion of the plurality of detection zones when the polymer is in a different position within the detection zones. As used herein, “plurality of detection zones” can refer to up to 2 detection zones, up to 50 detection zones, up to 1000 detection zones, or even up to more than 1000 detection zones. The detection signals associated with each of the detection zones may be combined such that the resulting combined detection signal exhibits both improved signal-to-noise ratio and increased effective sampling rate.

In one illustrative embodiment, the plurality of detection zones may comprise a first set of detection zones and a second set including the remaining detection zones. Parameters of the system may be set such that a polymer passing through the plurality of detection zones is in a substantially similar position in each detection zone of the first set when emissions are sampled. That is, if a polymer is just entering one detection zone of the first set at the end of a sample interval, it will also be just entering other detection zones of the first set at the end of other sample intervals, although this may not be the case for all sample intervals. The polymer may also be positioned in each detection zone of the second set as emissions are sampled, but be in a different position within the zones of the first and second set as emissions are sampled. In this regard, when detection signals associated with the first set of zones are combined with one another, the signal-to-noise ratio of the combined signal is improved. A similar effect occurs when detection signals associated with the second set are combined with one another. When signals of the first and second set are combined with one another, the effective sampling rate of the detection signal may be increased by a factor of two. In this regard, this embodiment benefits from multiple aspects of the invention.

In the above described embodiment, emissions of a polymer are sampled from a series of detection zones such that the polymer is positioned substantially similarly within some of the series of zones. In this particular embodiment, the polymer is positioned substantially similarly in every other detection zone. However, in other embodiments, the polymer may be positioned substantially similarly in every third or fourth detection zone, or any other number, as the invention is not limited in this regard. In some embodiments, the detection system may be constructed such that the transit interval is set equal to the sample interval multiplied by a constant value (N), as represented by Eq. 1 below. Here, Eq. 1 characterizes the relationship between the transit interval and the sample interval. Specifically, Eq. 1 identifies the number of sample intervals that occur within a given transit interval. Systems having the relationship between transit interval and sample interval defined by Eq. 1, where N is an integer, will have a polymer in a substantially similar position in each of their detection zones—absent altering other system factors.
Transit Interval=N*Sample Interval Eq. 1

In other embodiments, a constant (delta) may be included in the equation that defines the relationship between transit interval, sample interval, and N, as reflected by Eq. 2. Embodiments that can be characterized by Eq. 2, where delta has a positive non-zero integer value, may have a polymer positioned substantially similarly in some of the detection zones. In particular, the polymer will be periodically found in substantially similar positions, at least in embodiments having a row of adjacent detection zones like that shown in FIG. 2. In these cases, Eq. 3 may be used to evaluate how frequently the polymer will be positioned substantially similarly within detection zones as emissions are sampled. In particular, (T) in Eq. 3 reflects the period of such relationships.
Transit Interval=N*Sample Interval+delta Eq. 2
T=Transit Interval/delta Eq. 3

It is to be appreciated that embodiments of the invention may be characterized where the period (T) of Eq. 3 takes on a non-integer value. In such embodiments, polymers may not appear in substantially similar positions in different detection zones on a periodic basis. This may be desired in some embodiments, particularly those that are primarily focused on increasing the effective sampling rate.

Various aspects of the invention involve combining different detection signals to improve polymer analysis. Many approaches involve first aligning the detection signals with one another and then summing the data points of each detection signal with one another. However, as is to be appreciated, the methods used to combine detection signals are not limited to those that involve aligning the detection signals and then summing them in any particular fashion.

Detection signals may be aligned with one another by shifting one with respect to another in either temporal or spatial domains. As discussed herein, many embodiments of the present invention include multiple detection zones arranged in an array, where a polymer to be analyzed is passed through detection zones of the array in a serial manner. The detection signals created in such an embodiment are aligned such that portions of the detection signals associated with corresponding portions of a polymer are also aligned with one another. In some embodiments, this may be accomplished by shifting one of the detection signals by a time or distance equal to the transit interval between the corresponding adjacent detection zones. As is to be appreciated, this may occur in either temporal or spatial domains. The term “phase distance” is used herein to describe the distance that a signal may be shifted in the spatial domain to align the signal with another detection signal. Phase distance is typically equal to the transit interval between adjacent detection zones. Elapsed time is the term used to describe the amount of time that a detection signal is shifted to bring it into alignment with another detection signal. Similarly, alignment of detection signals may be accomplished either by adding time or position to a detection signal associated with a zone that the polymer passes through first, or by subtracting time or position from a detection signal associated with a detection zone that the next polymer passes through.

In some illustrative embodiments, detection signals may be aligned with one another by first identifying and then aligning common characteristics within the detection signals. For example, emissions from a polymer, such as from a labeled probe bound to a repetitive sequence or an origin of replication or a centromere may be used to identify a point that is common within the various different detection signals and which represents a common portion of the polymer being analyzed. For instance, emissions from a polymer such as from an intercalating dye that illuminates the backbone of the polymer such as human intercalating dye that illuminates the backbone of the polymer, may be used to identify a midpoint of the polymer or either of its ends. Once a common feature or features are identified in multiple detection signals, the detection signals may then be aligned by shifting one of the detection signals with respect to another until the common feature is aligned. The detection signals may then be added, or averaged, according to aspects of the present invention.

In one illustrative embodiment of the invention, the detection signals and corresponding detection zones may be used to identify the relative velocity between a polymer and the detection zones. In some of such embodiments, two of a plurality of detection zones may be used to identify when a polymer enters the first zone and the time that elapses before the polymer enters the second zone. Knowledge of the elapsed time, and the distance between the first and second zones can then be used to identify the average velocity between the two zones. As is to be appreciated, in some embodiments, these detection zones may be dedicated initial and final timing detection zones. However, in other embodiments, detection signals from zones also used in other aspects of the analysis may be used to quantify velocity, as the invention is not limited in this respect.

Numerous types of detectors exist and may be used in embodiments of detections systems according to the present invention. In one illustrative embodiment of an optical detection system, avalanche photo diodes may be used to detect photons that are emitted from labels or other elements within a detection zone. The photon counts, or lack thereof, may be used to determine whether a particular label is present on a polymer at a given time. In some embodiments, detection signals may be directed to a detector comprising a Charge Coupled Device (CCD), where the photon intensity may be detected in the various pixels of the CCD. Such pixels may be arranged in a two dimensional array, or in a linear array of the CCD device. It is to be appreciated that the present invention is not limited to any specific type of detector, and that the above described detectors are merely examples.

CCD detectors and Complimentary Metal Oxide Semiconductor detectors (CMOS) are examples of wide-field imaging devices that may be used as detectors within embodiments of the present invention. A CCD is an array of photosensitive elements, where each element is capable of generating an electrical response to photons that are incident upon it. Each element may be referred to as a pixel and is typically a square having side dimensions between 20 and 30 microns, although it is not so limited. The pixels of the CCD collect photons that are incident upon them and convert them to electrical charges representative of the number of photons counted. The charges are then passed along a first direction of the two-dimensional array of pixels until all of the charges are represented in a single linear array of the CCD. After all of the counts are collected in this single array, they are passed into a corner of the two-dimensional array (i.e., an end of the linear array) where they may be passed, in turn, to the data processor. The data processor interprets the signal provided by the CCD and may reconstruct it as an array representing photon counts at each of the pixels over the entire area of the CCD. As may be appreciated, the processing time for a detection system that uses a CCD, or other type of wide field imaging device, may be substantially greater than a system that uses a point detector due to the additional, above-described processing steps. It is to be appreciated that although a CCD has been discussed as an exemplary wide field imaging device, other devices known to those in the art, such as CMOS detectors and others may also be used.

The methods of the invention can be used to generate information about naturally or non-naturally occurring molecules such as naturally or non-naturally occurring polymers. Preferably such polymers are nucleic acids. This information is generally based on signals arising from the binding of probes to target polymers. In some instances, the information is unit specific information which refers to any structural information about one, some, or all of the units that make up the polymer. If the polymer is a nucleic acid, the units are single or combinations of nucleotides, preferably arranged contiguously. The structural information obtained by analyzing a polymer may include the identification of its characteristic properties which (in turn) allows for, for example, the identification of its presence in or absence from a sample, determination of the relatedness of more than one polymers, identification of the size of the polymer, determination of the order, proximity or distance between two or more individual units within a polymer, and/or identification of the general composition of the polymer. Since the structure and function of polymers can be interdependent, structural information can reveal important information about the function of the polymer.

The sensitivity of methods provided herein allows polymers such as nucleic acids to be analyzed individually. Thus, the term “analyzing a polymer” as used herein means obtaining some information about the structure of the polymer such as its size, the order of its units, its relatedness to other molecules, the identity of its units, or its presence or absence in a sample. Analyzing the polymer generally requires contacting the polymer with a probe and determining the binding pattern of the probe to the polymer. As stated herein, such binding patterns may simply indicate if the probe is bound to the polymer. Alternatively, the binding pattern may represent all of a portion of sites on the polymer to which the probe has bound. In this respect, the binding pattern can provide a map of sites along the polymer. Emission levels as well as emission positions may therefore be analyzed.

Analyzing a polymer applies to analyzing a nucleic acid, a peptide, a protein, a polysaccharide, and the like. It is to be understood that the same definitions apply to non-naturally occurring molecules such as non-naturally occurring polymers. The polymer being analyzed is referred to as the “target polymer”. The nucleic acid being analyzed is referred to as the nucleic acid target.

A “polymer” as used herein is a compound having contiguous individual units which are linked together at a backbone. In some cases, the polymer may be branched. Preferably the polymer is unbranched. The term “backbone” is given its usual meaning in the field of polymer chemistry. The polymers may be heterogeneous in unit and backbone composition.

The term “nucleic acid” refers to multiple linked nucleotides (i.e., molecules comprising a sugar (e.g., ribose or deoxyribose) linked to an exchangeable organic base, which is either a pyrimidine (e.g., cytosine (C), thymidine (T) or uracil (U)) or a purine (e.g., adenine (A) or guanine (G)). “Nucleic acid” and “nucleic acid molecule” are used interchangeably and refer to oligoribonucleotides as well as oligodeoxyribonucleotides. The terms shall also include polynucleosides (i.e., a polynucleotide minus a phosphate) and any other organic base containing nucleic acid. The nucleic acids may be single or double stranded. The size of the nucleic acid is not critical to the invention and it is generally only limited by the detection system used.

The invention can be applied to various forms of nucleic acids including DNAs and RNAs. Examples of DNAs include genomic DNA, such as nuclear DNA, and mitochondrial DNA, and cDNA. Examples of RNAs include but are not limited to messenger RNA (mRNA), ribosomal RNA (rRNA), microRNA (miRNA), small interfering RNA (siRNA), and the like. MicroRNA is a class of noncoding RNAs generally about 22 nucleotides in size that are believed involved in the regulation of gene expression. siRNA is a double stranded RNA involved in RNA interference. siRNA reportedly induces the formation of a ribonucleoprotein complex, which in turn mediates sequence-specific cleavage of a transcript target. It is to be understood that miRNA and siRNA can be used as either targets or as probes in the invention. The invention can also be applied to non-naturally occurring nucleic acids such as those containing peptide-nucleic acid (PNA) or locked-nucleic acid (LNA) elements. Such nucleic acids can be targets and/or probes.

In some preferred embodiments, the nucleic acid is directly harvested and isolated from a biological sample (such as a tissue or a cell culture). Harvest and isolation of nucleic acids are routinely performed in the art and suitable methods can be found in standard molecular biology textbooks. (See, for example, Maniatis' Handbook of Molecular Biology.) The nucleic acid may be harvested from a biological sample such as a tissue or a biological fluid. The term “tissue” as used herein refers to both localized and disseminated cell populations including, but not limited, to brain, heart, breast, colon, bladder, uterus, prostate, stomach, testis, ovary, pancreas, pituitary gland, adrenal gland, thyroid gland, salivary gland, mammary gland, kidney, liver, intestine, spleen, thymus, bone marrow, trachea, and lung. Biological fluids include saliva, sperm, serum, plasma, blood and urine, but are not so limited. Both invasive and non-invasive techniques can be used to obtain such samples and are well documented in the art.

The methods of the invention may be performed in the absence of prior nucleic acid amplification in vitro. Accordingly, some embodiments of the invention involve analysis of “non in vitro amplified nucleic acids”. As used herein, a “non in vitro amplified nucleic acid” refers to a nucleic acid that has not been amplified in vitro using techniques such as polymerase chain reaction or recombinant DNA methods. A non in vitro amplified nucleic acid may, however, be a nucleic acid that is amplified in vivo (e.g., in the biological sample from which it was harvested) as a natural consequence of the development of the cells in the biological sample. This means that the non in vitro nucleic acid may be one which is amplified in vivo as part of gene amplification, which is commonly observed in some cell types as a result of mutation or cancer development.

In some embodiments, the invention embraces nucleic acid derivatives as targets and/or probes. As used herein, a “nucleic acid derivative” is a non-naturally occurring nucleic acid. Nucleic acid derivatives may contain non-naturally occurring elements such as non-naturally occurring nucleotides and non-naturally occurring backbone linkages. These include substituted purines and pyrimidines such as C-5 propyne modified bases, 5-methylcytosine, 2-aminopurine, 2-amino-6-chloropurine, 2,6-diaminopurine, hypoxanthine, 2-thiouracil and pseudoisocytosine. Other such modifications are well known to those of skill in the art.

The nucleic acid derivatives may also encompass substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Nucleic acid derivatives may include a 2′-O-alkylated ribose group and/or sugars such as arabinose instead of ribose.

The nucleic acids may be heterogeneous in backbone composition thereby containing any possible combination of nucleic acid units linked together such as peptide nucleic acids (which have amino acid linkages with nucleic acid bases, and which are discussed in greater detail herein). In some embodiments, the nucleic acids are homogeneous in backbone composition.

As used herein with respect to linked units of a nucleic acid, “linked” or “linkage” means two entities bound to one another by any physicochemical means. Any linkage known to those of ordinary skill in the art, covalent or non-covalent, is embraced. Natural linkages, which are those ordinarily found in nature connecting the individual units of a particular nucleic acid, are most common. Natural linkages include, for instance, amide, ester and thioester linkages. The individual units of a nucleic acid analyzed by the methods of the invention may be linked, however, by synthetic or modified linkages. Nucleic acids where the units are linked by covalent bonds will be most common but those that include hydrogen bonded units are also embraced by the invention. It is to be understood that all possibilities regarding nucleic acids apply equally to nucleic acid targets and nucleic acid probes.

A nucleic acid target can be bound by one or more sequence-specific probes. “Sequence-specific” when used in the context of a probe for a nucleic acid target means that the probe recognizes a particular contiguous arrangement of nucleotides or derivatives thereof. In preferred embodiments, the probe is itself composed of nucleic acid elements such as DNA, RNA, PNA and LNA elements and combinations thereof (as discussed below). In preferred embodiments, the linear arrangement includes contiguous nucleotides or derivatives thereof that each bind to a corresponding complementary nucleotide in the probe. In some embodiments, however, the sequence may not be contiguous as there may be one, two, or more nucleotides that do not have corresponding complementary residues on the probe. The specificity of binding can be manipulated in a number of ways including temperature, salt concentration and the like. Those of ordinary skill in the art will be able to determine optimum conditions for a desired specificity.

It is to be understood that any molecule that is capable of recognizing a target nucleic acid with structural or sequence specificity can be used as a nucleic acid probe. In most instances, such probes will be themselves nucleic acid in nature. Also in most instances, such probes will form at least a Watson-Crick bond with the nucleic acid target. In other instances, the nucleic acid probe can form a Hoogsteen bond with the nucleic acid target, thereby forming a triplex. A nucleic acid probe that binds by Hoogsteen binding enters the major groove of a nucleic acid target and hybridizes with the bases located there. Examples of these latter probes include molecules that recognize and bind to the minor and major grooves of nucleic acids (e.g., some forms of antibiotics). In some embodiments, the nucleic acid probes can form both Watson-Crick and Hoogsteen bonds with the nucleic acid target. Bis PNA probes, for instance, are capable of both Watson-Crick and Hoogsteen binding to a nucleic acid.

In some embodiments, the nucleic acid probe is a peptide nucleic acid (PNA), a bis PNA clamp, a pseudocomplementary PNA, a locked nucleic acid (LNA), DNA, RNA, or co-nucleic acids of the above such as DNA-LNA co-nucleic acids. In some instances, the nucleic acid target can also be comprised of any of these elements.

PNAs are DNA analogs having their phosphate backbone replaced with 2-aminoethyl glycine residues linked to nucleotide bases through glycine amino nitrogen and methylenecarbonyl linkers. PNAs can bind to both DNA and RNA targets by Watson-Crick base pairing, and in so doing form stronger hybrids than would be possible with DNA or RNA based probes.

PNAs are synthesized from monomers connected by a peptide bond (Nielsen, P. E. et al. Peptide Nucleic Acids, Protocols and Applications, Norfolk: Horizon Scientific Press, p. 1-19 (1999)). They can be built with standard solid phase peptide synthesis technology. PNA chemistry and synthesis allows for inclusion of amino acids and polypeptide sequences in the PNA design. For example, lysine residues can be used to introduce positive charges in the PNA backbone. All chemical approaches available for the modifications of amino acid side chains are directly applicable to PNAs.

Several types of PNA designs exist, and these include single strand PNA (ssPNA), bis PNA and pseudocomplementary PNA (pcPNA).

The structure of PNA/DNA complex depends on the particular PNA and its sequence. Single stranded PNA (ssPNA) binds to single stranded DNA (ssDNA) preferably in antiparallel orientation (i.e., with the N-terminus of the ssPNA aligned with the 3′ terminus of the ssDNA) and with a Watson-Crick pairing. PNA also can bind to DNA with a Hoogsteen base pairing, and thereby forms triplexes with double stranded DNA (dsDNA) (Wittung, P. et al., Biochemistry 36:7973 (1997)).

Single strand PNA is the simplest of the PNA molecules. This PNA form interacts with nucleic acids to form a hybrid duplex via Watson-Crick base pairing. The duplex has different spatial structure and higher stability than dsDNA (Nielsen, P. E. et al. Peptide Nucleic Acids, Protocols and Applications, Norfolk: Horizon Scientific Press, p. 1-19 (1999)). However, when different concentration ratios are used and/or in presence of complimentary DNA strand, PNA/DNA/PNA or PNA/DNA/DNA triplexes can also be formed (Wittung, P. et al., Biochemistry 36:7973 (1997)).

Bis PNA includes two strands connected with a flexible linker. One strand is designed to hybridize with DNA by a classic Watson-Crick pairing, and the second is designed to hybridize with a Hoogsteen pairing. The target sequence can be short (e.g., 8 bp), but the bis PNA/DNA complex is still stable as it forms a hybrid with twice as many (e.g., a 16 bp) base pairings overall. The bis PNA structure further increases specificity of their binding. As an example, binding to an 8 bp site with a probe having a single base mismatch results in a total of 14 bp rather than 16 bp.

Pseudocomplementary PNA (pcPNA) (Izvolsky, K. I. et al., Biochemistry 10908-10913 (2000)) involves two single stranded PNAs added to dsDNA. One pcPNA strand is complementary to the target sequence, while the other is complementary to the displaced DNA strand. As the PNA/DNA duplex is more stable, the displaced DNA generally does not restore the dsDNA structure.

Locked nucleic acid (LNA) is a modified RNA nucleotide. Synthesis and hybridization profiles are described by Braasch and Corey (Chem. Biol. 2001 January; 8(1): 1-7. Review). Commercial nucleic acid synthesizers and standard phosphoramidite chemistry may be used to make LNA.

Commercial nucleic acid synthesizers and standard phosphoramidite chemistry are used to make LNAs. Therefore, production of mixed LNA/DNA sequences is as simple as that of mixed PNA/peptide sequences.

The probes can also be stabilized in part by the use of other backbone modifications. The invention intends to embrace, in addition to the peptide and locked nucleic acids discussed herein, the use of the other backbone modifications such as but not limited to phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

Other backbone modifications include acetyl caps, amino spacers such as O-linkers, amino acids such as lysine (particularly useful if positive charges are desired in the PNA), and the like. Various PNA modifications are known and probes incorporating such modifications are commercially available from sources such as Boston Probes, Inc.

The length of probe can also determine the specificity of binding.

The nucleic acid probes of the invention can be any length ranging from at least 4 nucleotides long to in excess of 1000 nucleotides long. In preferred embodiments, the probes are 5-100 nucleotides in length, more preferably between 5-25 nucleotides in length, and even more preferably 5-12 nucleotides in length. The length of the probe can be any length of nucleotides between and including the ranges listed herein, as if each and every length was explicitly recited herein. Thus, the length may be at least 5 nucleotides, at least 10 nucleotides, at least 15 nucleotides, at least 20 nucleotides, or at least 25 nucleotides. It should be understood that not all residues of the probe need to hybridize to complementary residues in the nucleic acid target. For example, the probe may be 50 residues in length, yet only 25 of those residues hybridize to the nucleic acid target. Preferably, the residues that hybridize are contiguous with each other. Similarly, the probe and any nucleic acids to which it binds need not be of the same size.

The probes are preferably single stranded, but they are not so limited. For example, when the probe is a bis PNA it can adopt a secondary structure with the nucleic acid target resulting in a triple helix conformation, with one region of the bis PNA clamp forming Hoogsteen bonds with the backbone of the target and another region of the bis PNA clamp forming Watson-Crick bonds with the nucleotide bases of the target.

The nucleic acid probe hybridizes to a complementary sequence within the nucleic acid target. The specificity of binding can be manipulated based on the hybridization conditions. For example, salt concentration and temperature can be modulated in order to vary the range of sequences recognized by the nucleic acid probes.

Polymers can be labeled using antibodies or antibody fragments and their corresponding antigen or hapten binding partners. Detection of such bound antibodies and proteins or peptides is accomplished by techniques well known to those ordinarily skilled in the art. Antibody/antigen complexes are easily detected by linking a label to the antibodies which recognize the polymer and then observing the site of the label. Alternatively, the antibodies can be visualized using secondary antibodies or fragments thereof that are specific for the primary antibody used. Polyclonal and monoclonal antibodies may be used. Antibody fragments include Fab, F(ab)₂, Fd and antibody fragments which include a CDR3 region.

The various reagents, reactive groups, and probes may in some instances include a linker molecule. These linkers can be any variety of molecules, preferably non-active, such as nucleotides or multiple nucleotides, straight or branched saturated or unsaturated chains of carbon, phospholipids, and the like, whether naturally occurring or synthetic. Additional linkers include alkyl and alkenyl carbonates, carbamates, and carbamides.

A wide variety of linkers can be used, many of which are commercially available, for example, from sources such as Boston Probes, Inc. (now Applied Biosystems, Inc.). Linkers are not limited to organic linkers, and rather can be inorganic also (e.g., —O—Si—O—, or O—P—O—). Additionally, they can be heterogeneous in nature (e.g., composed of organic and inorganic elements). Essentially any molecule having the appropriate size restrictions and capable of being linked to the various components such as fluorophore and probe can be used as a linker. As used herein, the terms linker and spacer are used interchangeably.

A “polymer dependent impulse” as used herein is a detectable physical quantity which transmits or conveys information about the structural characteristics of a unit of a polymer. The physical quantity may be in any form which is capable of being detected. For instance the physical quantity may be electromagnetic radiation, chemical conductance, electrical conductance, etc. The polymer dependent impulse may arise from energy transfer, quenching, changes in conductance, radioactivity, mechanical changes, resistance changes, or any other physical changes.

The method used for detecting the polymer dependent impulse depends on the type of physical quantity generated. For instance if the physical quantity is electromagnetic radiation, then the polymer dependent impulse is optically detected. An “optically detectable” polymer dependent impulse as used herein is a light based signal in the form of electromagnetic radiation which can be detected by light detecting imaging systems. In some embodiments the intensity of this signal is measured. When the physical quantity is chemical conductance, then the polymer dependent impulse is chemically detected. A “chemically detected” polymer dependent impulse is a signal in the form of a change in chemical concentration or charge such as ion conductance which can be detected by standard means for measuring chemical conductance. If the physical quantity is an electrical signal, then the polymer dependent impulse is in the form of a change in resistance or capacitance. These types of signals and detection mechanisms are described in U.S. Pat. No. 6,355,420 B1.

A detectable label is a moiety, the presence of which can be ascertained directly or indirectly. Generally, detection of the label involves an emission of energy by the label. The label can be detected directly for example by its ability to emit and/or absorb electromagnetic radiation of a particular wavelength. A label can be detected indirectly by its ability to bind, recruit and, in some cases, cleave another moiety which itself may emit or absorb light of a particular wavelength (e.g., an epitope tag such as the FLAG epitope, an enzyme tag such as horseradish peroxidase, etc.).

It is to be understood that a polymer that is said to “have” a label or a polymer with labels “disposed thereon” is a polymer that may have a label intrinsically as a part of the polymer. It is also to be understood that a polymer that is said to “have” a label or a polymer with labels “disposed thereon” may be a polymer that is bound to an extrinsic element such as a probe that comprises the label, such as a fluorophor, a radio opaque marker, and the like.

Generally, a detectable label can be but is not limited to a chromogenic molecule, a fluorescent molecule (e.g., fluorescein isothiocyanate (FITC), TRITC, rhodamine, tetramethylrhodamine, R-phycoerythrin, Cy-3, Cy-5, Cy-7, Texas Red, Phar-Red and allophycocyanin (APC)), a chemiluminescent molecule, a bioluminescent molecule, a radioisotope (e.g., P³² or H³, ¹⁴C, ¹²⁵I and ¹³¹I), an optical or electron density molecule, an electromagnetic molecule, an electrical charge transducing or transferring molecule, a semiconductor nanocrystal or nanoparticle, an electron spin resonance molecule (such as for example nitroxyl radicals), a nuclear magnetic resonance molecule, a colloidal metal, a colloid gold nanocrystal, a microbead, a magnetic bead, a paramagnetic particle, a quantum dot, an enzyme (e.g., alkaline phosphatase, horseradish peroxidase, β-galactosidase, glucoamylase, lysozyme, luciferases such as firefly luciferase and bacterial luciferase (U.S. Pat. No. 4,737,456); saccharide oxidases such as glucose oxidase, galactose oxidase, and glucose-6-phosphate dehydrogenase; heterocyclic oxidases such as uricase and xanthine oxidase coupled to an enzyme that uses hydrogen peroxide to oxidize a dye precursor such as HRP, lactoperoxidase, or microperoxidase), an enzyme substrate, an affinity molecule, a ligand, a receptor, a biotin molecule, an avidin molecule, a streptavidin molecule, an antigen (e.g., epitope tags such as the FLAG or HA epitope), a hapten (e.g., biotin, pyridoxal, digoxigenin fluorescein and dinitrophenol), an antibody and an antibody fragment. The label may be of a chemical, lipid, carbohydrate, peptide or nucleic acid nature although it is not so limited. Those of ordinary skill in the art will know of other suitable labels for the binding assay components (or therapeutic agents described herein), or will be able to ascertain such information using routine experimentation.

The detection system can be selected from any number of detection systems known in the art. These include a charge coupled device (CCD) detection system, an electron spin resonance (ESR) detection system, an electrical detection system, an electron microscopy detection system, a confocal laser microscopy detection system, a photographic film detection system, a fluorescent detection system, a chemiluminescent detection system, an enzyme detection system, an atomic force microscopy (AFM) detection system, a scanning tunneling microscopy (STM) detection system, a scanning electron microscopy detection system, an optical detection system, an electron density detection system, a refractive index system, a nuclear magnetic resonance (NMR) detection system, a near field detection system, a total internal reflection (TIR) detection system, and an electromagnetic detection system.

The label may be bound to probe during or following its synthesis. As used herein, “conjugated” means two entities stably bound to one another by any physiochemical means. It is important that the nature of the attachment is such that it does not substantially impair the effectiveness of either entity. Keeping these parameters in mind, any covalent or non-covalent linkage known to those of ordinary skill in the art may be employed. In some embodiments, covalent linkage is preferred. Noncovalent conjugation includes hydrophobic interactions, ionic interactions, high affinity interactions such as biotin-avidin and biotin-streptavidin complex formation and other affinity interactions. Such means and methods of attachment are known to those of ordinary skill in the art. Furthermore, the coupling or conjugation of these labels to the binding assay components of the invention can be performed using standard techniques common to those of ordinary skill in the art. For example, U.S. Pat. Nos. 3,940,475 and 3,645,090 demonstrate conjugation of fluorophores and enzymes to antibodies.

In some embodiments, more than one labeled probe is used and the close association of those probes (due to proximate specific binding) results in the absence or the presence of a signal. One common example of this configuration is fluorescence resonance energy transfer (FRET). In FRET, one probe is labeled with an donor molecule that accepts light of a certain wavelength and emits light of another. A second probe is labeled with an acceptor molecule that accepts light at the wavelength emitted by the donor molecule and emits light at a different wavelength. The donor molecule however can only impart its emitted light if it is in close enough proximity to the acceptor molecule. The binding of two FRET labeled probes therefore is indicated by the emission of light from the acceptor molecule or the loss of emission from the donor molecule.

The methods provided herein are capable of generating signatures for each polymer based on the specific binding patterns of probes to polymers. A signature is the binding pattern of the binding of probes along the length of the polymer. The signature of the polymer uniquely identifies the polymer.

In one embodiment, analysis of the polymer involves detecting signals from the labels (potentially through the use of a secondary label, as the case may be), and determining the relative position of those labels relative to one another. In some instances, it may be desirable to further label the polymer with a standard marker that facilitates comparing the information so obtained with that from other polymers analyzed. For example, the standard marker may be a backbone label, or a label that binds to a particular sequence of nucleotides (be it a unique sequence or not), or a label that binds to a particular location in the nucleic acid molecule (e.g., an origin of replication, a transcriptional promoter, a centromere, etc.).

One subset of backbone labels for nucleic acids are nucleic acid stains that bind nucleic acids in a sequence independent manner. Examples include intercalating dyes such as phenanthridines and acridines (e.g., ethidium bromide, propidium iodide, hexidium iodide, dihydroethidium, ethidium homodimer-1 and -2, ethidium monoazide, and ACMA); minor groove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc. Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red).

Detectable signals are generated, such as optical signals or other types, and are detected and stored in a database. The signals can be analyzed to determine structural information about the polymer. The signals can be analyzed by assessing the intensity of the signal to determine structural information about the polymer. The computer may be the same computer used to collect data about the polymers, or may be a separate computer dedicated to data analysis. A suitable computer system to implement embodiments of the present invention typically includes an output device which displays information to a user, a main unit connected to the output device and an input device which receives input from a user. The main unit generally includes a processor connected to a memory system via an interconnection mechanism. The input device and output device also are connected to the processor and memory system via the interconnection mechanism. Computer programs for data analysis of the detected signals are readily available from CCD (Charge Coupled Device) manufacturers.

Once all of the detectable signals are generated, detected and stored in a database the signals can be analyzed to determine structural information about the polymer. The computer may be the same computer used to collect data about the polymers, or may be a separate computer dedicated to data analysis. A suitable computer system to implement the present invention typically includes an output device which displays information to a user, a main unit connected to the output device and an input device which receives input from a user. The main unit generally includes a processor connected to a memory system via an interconnection mechanism. The input device and output device also are connected to the processor and memory system via the interconnection mechanism.

It should be understood that one or more output devices may be connected to the computer system. Example output devices include a cathode ray tube (CRT) display, liquid crystal displays (LCD), printers, communication devices such as a modem, and audio output. It should also be understood that one or more input devices may be connected to the computer system. Example input devices include a keyboard, keypad, track ball, mouse, pen and tablet, communication device, and data input devices such as sensors. It should be understood the invention is not limited to the particular input or output devices used in combination with the computer system or to those described herein.

The computer system may be a general purpose computer system which is programmable using a high level computer programming language, such as C or C++. The computer system may also be specially programmed with special purpose hardware. In a general purpose computer system, the processor is typically a commercially available processor, of which the series x86 processors, available from Intel, and similar devices from AMD and Cyrix, the 680X0 series microprocessors available from Motorola, the PowerPC microprocessor from IBM and the Alpha-series processors from Digital Equipment Corporation, are examples. Many other processors are available. Such a microprocessor executes a program called an operating system, of which WindowsNT, UNIX, DOS, VMS, LINUX, and OSX are examples, which controls the execution of other computer programs and provides scheduling, debugging, input/output control, accounting, compilation, storage assignment, data management and memory management, and communication control and related services. The processor and operating system define a computer platform for which application programs in high-level programming languages are written.

A memory system typically includes a computer readable and writeable nonvolatile recording medium, of which a magnetic disk, a flash memory and tape are examples. The disk may be removable, known as a floppy disk, or permanent, known as a hard drive. A disk has a number of tracks in which signals are stored, typically in binary form, i.e., a form interpreted as a sequence of one and zeros. Such signals may define an application program to be executed by the microprocessor, or information stored on the disk to be processed by the application program. Typically, in operation, the processor causes data to be read from the nonvolatile recording medium into an integrated circuit memory element, which is typically a volatile, random access memory such as a dynamic random access memory (DRAM) or static memory (SRAM). The integrated circuit memory element allows for faster access to the information by the processor than does the disk. The processor generally manipulates the data within the integrated circuit memory and then copies the data to the disk when processing is completed. A variety of mechanisms are known for managing data movement between the disk and the integrated circuit memory element, and the invention is not limited thereto. It should also be understood that the invention is not limited to a particular memory system.

It should be understood the invention is not limited to a particular computer platform, particular processor, or particular high-level programming language. Additionally, the computer system may be a multiprocessor computer system or may include multiple computers connected over a computer network.

The data stored about the polymers may be stored in a database, or in a data file, in the memory system of the computer. The data for each polymer may be stored in the memory system so that it is accessible by the processor independently of the data for other polymers, for example by assigning a unique identifier to each polymer.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents, and patent applications that are recited in this application are hereby incorporated by reference in their entirety.

What is claimed is:

1. A method of analyzing at least one polymer, the method comprising the acts of:
providing the at least one polymer with one or more labels disposed thereon;
providing a plurality of detection zones and instrumentation adapted to detect emission signals from labels that pass through the detection zones, each of the detections zones having a zone distance between an upstream edge and a downstream edge;
passing the at least one polymer through at least a first and second detection zone of the plurality of detection zones at a velocity;
sampling emissions from the first detection zone at a first sample interval as the at least one polymer passes through the first detection zone to create a first detection signal;
sampling emissions from the second detection zone at a second sample interval as the at least one polymer passes through the second detection zone to create a second detection signal; and
combining the first and second detection signals together to create a combined signal used to analyze the at least one polymer.

2. The method of claim 1, further comprising:
sampling emissions from additional detection zones of the plurality of detection zones as the at least one polymer passes through the additional detection zones to create additional detection signals;
combining the additional detection signals with the first and second detection signals to create the combined signal used to analyze the at least one polymer.

3. (canceled)

4. The method of claim 1, wherein the at least one polymer is a single polymer.

5. The method of claim 1, wherein the at least one polymer is a plurality of polymers.

6. The method of claim 1, wherein any one of the at least one polymer is in a substantially similar position within each of the first and second detection zones when emissions are sampled.

7. The method of claim 6, wherein any one of the at least one polymer is in a substantially similar position by being an equal distance from the upstream edge of the first detection zone and the upstream edge of the second detection zone when emissions are sampled.

8. The method claim 6, wherein any one of the at least one polymer is in a substantially similar position due to either the first or second sample intervals being a factor of a transit interval between similar points within each of the first and second detection zones.

9. (canceled)

10. (canceled)

11. The method of claim 1, wherein any one of the at least one polymer is in a different position within each of the first and second detection zones when emissions are sampled.

12. The method of claim 11, wherein the first sample interval is different from the second sample interval such that any one of the at least one polymer is in a different position within each of the first and second detection zones when emissions are sampled.

13. (canceled)

14. (canceled)

15. The method of claim 11, wherein the first and second sample interval are defined by the velocity multiplied by a first and second acquisition time, respectively, and further wherein the first and second acquisition times are out of phase with one another such that any one of the at least one polymer is in a different position within each of the first and second detection zones when emissions are sampled.

16. The method claim 11, wherein a transit interval between similar points within each of the first and second detection zones is substantially equal to a multiple of either the first sample interval plus a constant or the second sample intervals plus a constant such that any one of the at least one polymer is positioned differently within each of the first and second detection zones when emissions are sampled.

17. (canceled)

18. (canceled)

19. The method of claim 16, further comprising:
sampling emissions from a third of the plurality of detection zones at a third sample interval as any one of the at least one polymer passes through the third detection zone to create a third detection signal, wherein any one of the at least one polymer is positioned substantially similarly within each of the first and third detection zones when emissions are sampled.

20. (canceled)

21. (canceled)

22. (canceled)

23. The method of claim 1, wherein each of the plurality of detection zones has a substantially similar zone distance.

24. The method of claim 1, wherein combining the first and second detection signals comprises:
aligning the first and second detection signals to one another; and
summing the first and second detection signals together to create the combined signal.

25. The method of claim 24, wherein aligning comprises identifying an elapsed time between when one of the at least one polymer enters the first and the second detection zones and shifting the second detection signal by an amount of time substantially equal to the elapsed time to align the first and second detection signals.

26. The method of claim 24, wherein aligning comprises calculating a phase distance between where one of the at least one polymer enters the first and the second detection zones and shifting the second detection signal by the phase distance to align the first and second detection signals.

27. (canceled)

28. The method of claim 24, wherein aligning the first and second detection signals comprises identifying a common element in each of the first and second detection signals and aligning the first and second detection signals by aligning the common element.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. A method for increasing a number of sampling points of a single polymer passing through an interaction area having a first and a second detection zone, the method comprising acts of:
sampling emissions from the first detection zone as the polymer passes there through to provide a first set of discrete sample points;
sampling emissions from the second detection zone as the polymer passes there through to provide a second set of discrete sample points; and
combining the first and second sets of discrete signal points to increase the number of sampling points of the polymer.

46. (canceled)

47. A computer readable medium having computer readable signals stored thereon that define instructions that, as a result of being executed by a computer, instruct the computer to perform a method of increasing a number of sampling points of a polymer passing through a sampling area, the method comprising acts of:
sampling emissions from the first detection zone as the polymer passes there through to provide a first set of discrete sample points;
sampling emissions from the second detection zone as the polymer passes there through to provide a second set of discrete sample points; and
combining the first and second sets of discrete signal points to increase the number of sampling points of the polymer.

48. (canceled)

49. An apparatus for analysis of a polymer, the apparatus comprising:
a microfluidic channel having a first and a second end, the microfluidic channel adapted to deliver a polymer disposed within a carrier fluid from the first to the second end;
an array of multiple detection zones disposed within the microfluidic channel and extending from the first end toward the second end, wherein the apparatus is adapted to detect emissions from the polymer as the polymer passes through the multiple detection zones to analyze the polymer.

50. (canceled)

51. (canceled)

52. (canceled)

53. (canceled)

54. (canceled)

55. (canceled)

56. (canceled)
